(19) United States
(12) Patent Application Publication
(10) Pub. No.: US 2007/0134350 A1
(43) Pub. Date: Jun. 14, 2007

(54) Oils/mixtures as deactivants

(75) Inventors: Sabrina Higgins, Southampton (GB); John Farrell Hughes, Southampton (GB); Malcolm Tom McKechnie, Hull (GB)

Correspondence Address: NORRIS, MCLAUGHLIN & MARCUS, 875 THIRD AVE, 18TH FLOOR, NEW YORK, NY, 10022, US

(73) Assignees: Reckitt Benckiser Limited, Slough, Berkshire (GB); University of Southhampton (CEI), Highfield, Southhampton (GB)

(21) Appl. No.: 10/595,767

(22) PCT Filed: Nov. 15, 2004
(86) PCT No.: PCT/GB04/04819

§ 371 Date: Sep. 5, 2006

(30) Foreign Application Priority Data
Nov. 15, 2003 (GB) 0326648.3

(51) Int. Cl. A61K 36/752; A61K 36/53; A61K 36/534; A61K 36/899; A61K 36/73; A61K 36/324

(52) U.S. Cl. 424/725; 424/736; 424/745; 424/747; 424/750; 424/765

(57) ABSTRACT

A method of deactivating an allergen comprises dispersing into an airspace containing or able to support said allergen an allergen-deactivating amount of an allergen-deactivating compound, the compound comprising one or more of the following compounds: a citrus oil; a mint oil; bois de rose oil; oil of jasmine; frankincense; oil of bergamot; and oil of lemon grass.

The present invention relates to a method of deactivating dust mite allergens.

Various allergens are known to trigger a human reaction. For example, it has been known for a long time that house dust can trigger allergenic reactions in humans, such as asthma and rhinitis. It was reported, as early as 1928 that it was the dust mites in the dust that were the primary source of the allergenic response, but it was only in the 1960's that researchers appreciated its significance.

House dust mites produce detritus which causes allergenic reaction in many people. The major allergens are believed to be Dermatophogoides farinae (known as Der f1) and Dermatophagoides pteronyssinus (known as Der p1), and to include faeces as well as body part residues of the house dust mites. A review is given in Experimental and Applied Acarology, 10 (1991) p. 167-186.

Other allergens which are problematic include cockroach allergens (notably the Bla g1 cockroach allergen). and cat allergens (Fel d1). In the case of cat allergens the pelt of the cat and/or its salivary deposits seem to be of significance in eliciting the allergenic response.

WO99/15208 describes a method for deactivating allergens derived from the D. Pteronyssinus and D. Farinae dust mite species, which comprises contacting the allergen with one of 28 deactivants which are described. These are chemically diverse. They include cyclodextrin, urea, hydrogenated hop oil, aluminium chlorohydrate and silica gel.

WO01/76371 describes further deactivants for house dust mite allergens. The further deactivants are cajeput oil (tea tree oil) and oils comprising one or more terpene hydrocarbons.

Clearly different types of compound may function as deactivants but there are still problems in finding deactivants which have high efficacy, and which are acceptable to consumers in a household environment. Firstly, most materials do not function as deactivants. Secondly, those that do often have an odour which consumers find unacceptable. Many of the deactivants described in WO99/15208 and WO01/76371 have odours which consumers find too pungent and/or to have too pronounced a “sanitary” or “antiseptic” quality. Thirdly prior methods and/or deactivants have sometimes caused staining of surfaces to which they are applied.

In accordance with a first aspect of the present invention there is provided a method of deactivating an allergen, the method comprising dispersing into an airspace containing or able to support said allergen an allergen-deactivating amount of an allergen-deactivating compound (hereinafter “deactivant”) selected from one or more of the following compounds:
a citrus oil;
a mint oil;
bois de rose oil;
oil of jasmine;
frankincense;
oil of bergamot; and
oil of lemon grass.

Such compounds have pleasant scents yet are effective in combating allergens.

A preferred deactivant is a mint oil, most preferably spearmint oil.

A preferred deactivant is oil of jasmine.

A preferred deactivant is frankincense.

An especially preferred deactivant is a citrus oil, most preferably orange oil. Other suitable citrus oils may include lemon oil, lime oil and grapefruit oil.

An especially preferred deactivant is bois de rose oil.

An especially preferred deactivant is oil of bergamot.

An especially preferred deactivant is oil of lemon grass.

A deactivant herein may be a significant separated component of any of the named oils; for example a distillation product thereof. In such a case the deactivant is preferably the largest component of the oil. Preferably, however, any oil referred to herein as the deactivant is in its as-extracted form. In particular, it has preferably not been distilled or otherwise treated in order to alter its chemical constitution or balance.

A preferred method employs two or more of the deactivants defined in the first aspect of the invention, preferably dispersed into the airspace simultaneously; most preferably having been mixed prior to dispersal. This can give deactivant activity in excess of that which would be predicted from the activity of each deactivant tested separately.

An especially preferred method employs oil of bergamot and bois de rose oil. An admixture thereof represents a further aspect of the present invention.

An especially preferred method employs oil of lemon grass and bois de rose oil. An admixture thereof represents a further aspect of the present invention.

Another preferred method employs a citrus oil and oil of bergamot. An admixture thereof represents a further aspect of the present invention.

An especially preferred method employs a citrus oil and oil of jasmine. An admixture thereof represents a further aspect of the present invention.

Preferably the allergen combated by one of the oils defined above is a Der p1 and/or Der f1 allergen.

The deactivant may suitably be dispersed into the airspace over an extended period, for example at least 30 minutes, and preferably at least 1 hour.

The deactivant may suitably be dispersed into the airspace on two occasions, interrupted by a period in which there is no deactivant dispersal. Deactivant may be dispersed into the airspace on one or more further occasions, following a corresponding period or periods of no deactivant dispersal. Preferably each such dispersal occasion involves deactivant dispersal over an extended period, as described above. Preferably the or each period in which there is no deactivant dispersal is an extended period, for example at least 2 hours, preferably at least 4 hours, and most preferably at least 8 hours. This repeated dispersal method appears to be particularly valuable when the deactivant is a citrus oil.

There are various methods which can be used to disperse the deactivant into the airspace. Examples are discussed in the passages below. In these passages the word deactivant is used to denote a single deactivant, and a plurality of deactivants used in a method of the invention, whether at the same time or at different times.

Preferably the deactivant is dispersed into the airspace as a vapour.

The deactivant may be vaporized by the use of heat. For example the deactivant, an oil, may be floated on water in an oil burner or heated directly in an oil burner. Alternatively the deactivant may be vaporized from a wick dipped into a reservoir of the deactivant. The wick may be burned, in the method.

Another method of dispersing the deactivant is by the ventilation of a source of the deactivant using an ion wind. An ion wind generates an ionized air flow which facilitates the evaporation and dispersal of the deactivant into the air. A unipolar charge is transferred to the molecules of the deactivant, which is evaporated. Optionally the source of the deactivant may be heated in order to assist evaporation. The ion wind not only facilitates the evaporation and dispersal of the deactivant but also has the added advantage that the ion wind generating device has no moving parts and thus operates at very low noise levels. The ion wind thus acts as an essentially silent fan. The charged molecules of the vaporized deactivant are attracted to particles in the air with an opposite or neutral charge and so may be more efficient at denaturing airborne allergens than uncharged molecules. The charged molecules are also attracted to surfaces in the environment which is being treated and thus allergens on surfaces are also treated.

It will be understood that in order to obtain the desired level of the deactivant evaporated into a room, the rate of evaporation of the deactivant will need to be taken into account, the surface area across which the deactivant is evaporated and the ion wind speed. Higher ion wind speeds will provide faster evaporation of the volatile components and thus the surface area across which the deactivant is evaporated will need to be adapted to the air flow speed.

The benefit of charging the molecules of the deactivant using an ion wind is two fold. The individual molecules are attracted as the allergen particles and, since all of the molecules have the same polarity charge, they are repelled one from another. Accordingly, the molecules tend to spread out to a great extent as compared to uncharged molecules.

Allergen particles are normally electrically isolated from their surroundings and will typically be at a potential which is the same as that of their surroundings. An isolated allergenic particle within a cloud of electrically charged molecules is likely to cause distortion of the electrical field so that the attraction of the charged molecules onto the allergen particle will be enhanced.

The deactivant may be used as such, or may be presented in the form of an emulsion. Generally, the emulsion will be an oil (i.e. deactivant)-in-water emulsion comprising up to 5% by weight of the deactivant (in total, when more than one of said deactivants is employed). The formation of emulsions is generally well known in the art and is described, for example, in Modern Aspects of Emulsion Science, edited by Bernard P. Binks, The Royal Society of Chemistry, 1998 and Surfactant Science and Technology, Second Edition, Drew Myers, 1992, VCH Publishers, Inc.

In a preferred aspect of the present invention a candle is used to promote the dispersal of the deactivant into the airspace. By the term “candle” as used herein is meant a solid, semi-solid or gelled body having a combustible body, which contains a wick which can carry a flame.

A candle may be located beneath a source of a deactivant, to accelerate its evaporation.

Alternatively or additionally the wick of a candle may deliver a deactivant by capillary action, from a source at one end of the candle, to be combusted at the other end.

Alternatively or additionally, and in any case preferably, a deactivant is incorporated into the combustible body of the candle.

A candle of use in the present invention preferably incorporates within its combustible body at least 2% by weight of the deactivant, preferably at least 5% by weight of the deactivant and more preferably at least 10% by weight of the deactivant (in total, when more than one of said deactivants is employed).

Typically, the combustible body of the candle may be a blend of organic materials such as beeswax, paraffin wax, montan wax, carnauba wax, microcrystalline wax, fatty alcohols, fatty acids, fatty esters or natural and synthetic resins. Clear candles may comprise as the combustible material a gel comprising mineral oil containing blends of diblock and triblock copolymers based on synthetic thermoplastic rubbers or a gel obtained by combining a liquid base material of a hydrogenated polyolefin, a gelling agent and optionally a gel enhancing agent.

A wick normally extends longitudinally through the candle body. More than one wick may be used, if desired, but usually a single wick is centrally disposed in the candle body. When a candle wick is ignited, the wick is adapted to burn gradually so that both the wick and the candle body are consumed.

Typically, the weight of candle which is burnt in a particular space to be treated will depend upon the actual volume of the space, e.g. room, to be treated.

The candle may suitably be burnt, and so its deactivant dispersed into the airspace, over an extended period, for example at least 1 hour, preferably at least 2 hours, and most preferably at least 5 hours.

The length of time for which the candle is burnt in the space to be treated will generally be for up to 2 hours, generally up to 5 hours, although in some circumstances the candle may be burnt for a longer period of time, such as 10 hours or more. However, it will be understood by those skilled in the art that an allergen denaturing effect will be obtained even if the candles containing the selected deactivants are burnt for a lesser period of time.

Another method of dispersing the deactivant is in the form of small droplets, preferably of mean diameter not exceeding 20 μm, preferably not exceeding 10 μm. Preferably such a method of dispersing small droplets is by use of an ultra-sonic jet nebuliser. The deactivant may be floated on the surface of water in the nebuliser, or provided as an oil (i.e. deactivant)-in-water emulsion in the nebuliser. The nebuliser may suitably comprise a piezo-ceramic element which vibrates in the liquid (at 2-5 MHz). A plume of liquid may be generated by ultrasonic streaming. A dense cloud of very small droplets (most preferably of mean diameter <5 μm) may then be expelled from the surface of the liquid.

A fan may be used to assist the expulsion of the nebulised droplets from the vessel.

The present invention involves the dispersal of an allergen deactivant into an airspace. It is possible that airborne allergens may be deactivated but it is believed that there is effective deactivation of allergens borne on surfaces within the airspace.

In accordance with a further aspect of the present invention there is provided the use in deactivating an allergen at a locus of one or more of the following materials:
a citrus oil;
a mint oil;
bois de rose oil;
oil of jasmine;
frankincense;
oil of bergamot;
oil of lemon grass.

In accordance with a further aspect of the present invention there is provided use of one or more of the following materials:
a citrus oil;
a mint oil;
bois de rose oil;
oil of jasmine;
frankincense;
oil of bergamot;
oil of lemon grass;
   dispersed into an airspace in order to deactivate an allergen on a surface which is within that airspace. Preferably the dispersal is as a vapour.

Preferably an allergen deactivated in a method or use in accordance with the present invention is a material which evokes an allergenic reaction in a human.

For example it may be an allergen arising from house dust mites, or from pets.

Most preferably the method or use of this invention is able to deactivate, partially or wholly, an allergen arising from the mite species Dermatophogoides farinae (known as Der f1) or, especially from the mite species Dermatophagoides pteronyssinus (known as Der p1).

The present invention will be further described with reference to the following Examples.

Experimental Protocol to Reduce Variability

When using house dust for allergen denaturing tests an inherent difficulty is the variability of the amount of allergen in each small sample, even when taken from the same dust reservoir. The amount of dust in the pre-treatment sample must be accurately estimated in order to determine the extent of any allergen denaturing. In these tests the dust sample was applied to the test exposure surface and then one half of this surface dust was removed to measure the control pre-treatment allergen level of that specific sample. Each control was directly relevant to each sample, which gave the best possible estimate of the level of allergen in the sample before exposure to possible denaturant. All tests employed a glass reinforced plastic booth of size 0.7 m×0.7 m×1.0 m. All tests had 5 or 6 replicates. Average values are stated.

The following Examples all measure the reduction of the house dust mite Dermatophagoides pteronyssinus allergen—Der p1.

### EXAMPLE 1—HARD SURFACE/OIL BURNER

House dust was passed through a number of sieves and the fraction smaller than 53 μm was collected. 0.025 g of dust was placed in a small sieve to distribute it evenly over the test surface. The test surface was a PTFE (polytetrafluoroethylene—trade mark TEFLON) coated metal tray of size 30 cm×30 cm. The dust was applied to the tray by moving the sieve continuously over the surface while tapping the sieve. One half of the dust was then removed by suction onto an in-line filter and the weight recorded, this was the pre-treatment control. The tray was then placed in the booth. An oil burner containing 800 μl of orange oil floated on 6 ml of distilled water was placed in the booth, and the booth was sealed. The oil burner candle was lit and allowed to burn until all the liquid had been vaporised (approx. 30 minutes). The candle was then smothered and the dust was left exposed in the booth. After 24 hours the tray was removed, the dust was collected from it and its weight recorded. The booth was washed with strong detergent between tests.

An identical test was carried out using water alone, with no orange oil.

The test samples were assayed for Der p1 using an ELISA (Enzyme linked immunosorbent assay) to determine the allergen content. This was then related to the weight of dust that had been present in each sample. All of the samples were multiplied up to compare the amount of allergen expected to be present in a 0.1 g sample of dust. The percentage difference between the control sample and the exposed sample was then obtained.

The sample exposed to orange oil vapour showed a reduction in Der p1 allergen content of the test dust samples of 97.9%. The water-only sample showed 23.8% reduction in allergen content.

### EXAMPLE 2—CARPET/OIL BURNER

House dust was passed through a number of sieves and the fraction smaller than 53 μm was collected. 0.1 g of dust was placed in a small sieve to distribute it evenly over the test surface. The test surface was a 30 cm² piece of polypropylene carpet. The dust was applied to the carpet by moving the sieve continuously over the surface. One half of the dust was then removed by suction onto an in-line filter and the weight recorded, this was the pre-treatment control. The carpet was then placed in the booth.

For control tests dust was distributed on a like carpet piece, the pre-treatment control was exposed to water alone (6.8 ml) in the oil burner and the dust was left in the booth for 24 hours. The carpet was then removed, the dust was collected from the carpet and weighed. An oil burner containing 800 μl of orange oil floated on 6 ml of distilled water was placed in the booth, and the booth was sealed. The oil burner candle was lit and allowed to burn until all the liquid had been vaporised (approx. 1 hour). The candle was then smothered and the dust was left exposed in the booth. After 16 hours the carpet was removed, the dust was collected from it and its weight recorded. The booth was washed with strong detergent between tests on the same chemical.

The test samples were assayed for Der p1 using an ELISA (Enzyme linked immunosorbent assay) to determine the allergen content. This was then related to the weight of dust that had been present in each sample. All of the samples were extrapolated up to compare the amount of allergen expected to be present in a 0.1 g sample of dust. The percentage difference between the control sample and the exposed sample was then obtained.

Treatment using water alone gave Der p1 allergen reduction of 13.9%. The orange oil gave a percentage reduction in the Der p1 allergen of 26.4%. When the test was repeated with a double dose of orange oil and water, together with water pre-treatment, the Der p1 allergen reduction was 94.6%.

### EXAMPLE 3—CARPET/CANDLE

Tests were carried out using candles loaded with deactivants. Test dust had been obtained from household vacuum cleaner bags. House dust was passed through a number of sieves and the fraction smaller than 53 μm was collected. 0.1 g of dust was placed in a small sieve to distribute it evenly over the test surface, a 30 cm² piece of polypropylene carpet. The dust was applied by moving the sieve continuously over the surface. Dust was removed from half of each test area by suction through an in-line glass fibre filter (2.5 cm diameter) and the weight recorded. Test candles were prepared, each of approximately 100 g before testing, with 2 wicks, and having a sterin:wax ratio of 2:10 (w/w) and 5% w/w of orange oil incorporated into the candle body, by a process of melting, mixing and setting. Candles were lit and placed in the respective booths for 5 hours. A repeat operation was carried out 16 hours is later with 6 ml water pre-treatment. In some cases a further repeat operation was carried out after a further period of 16 hours. The candles were then smothered and the dust was left exposed in the rooms for 16 hours. The dust was then collected as for the controls and weighed. An unfragranced candle was tested as a comparison.

During each 5 hour burn period approximately 27 g of each candle tested was burnt. This equated to a rate of 270 μl of deactivant released per hour.

The collected samples were assayed by Der p1 ELISA to determine the allergen content. This was then related to the weight of dust that had been present in each sample. All the samples were multiplied up to compare the amount of allergen expected to be present in a 0.1 g sample of dust. The percentage differences between the control samples and the exposed samples were then obtained.

Following 15 hours of burn time (3×5 hours) the unfragranced candle achieved a Der p1 allergen reduction of 36.3%. Following 10 hours of burn time (2×5 hours) the orange oil candle achieved a Der p1 allergen reduction of 70.0%. Following 15 hours of burn time (3×5 hours) the orange oil candle achieved a Der p1 allergen reduction of 87.6%.

### EXAMPLE 4—HARD SURFACE/CANDLE

Tests were carried out using candles loaded with deactivants. Test dust had been obtained from household vacuum cleaner bags. House dust was passed through a number of sieves and the fraction smaller than 53 μm was collected. 0.025 g of dust was placed in a small sieve to distribute it evenly over the test surface, a 30 cm×30 cm PTFE coated metal tray. The dust was applied by moving the sieve continuously over the surface. One half of the dust was then removed by suction onto an in-line filter and the weight recorded, this was the pre-treatment. The tray was then placed in the booth. Test candles were prepared, each of approximately 60 g before testing, with two wicks, and having a sterin:wax ration of 2:10 (w/w) and 5% w/w of deactivant incorporated into the candle body, by a process of melting, mixing and setting. The deactivants were:
orange oil
spearmint oil
bois de rose oil
frankincense
oil of bergamot
oil of lemon grass
bois de rose oil and oil of bergamot (2.5% w/w each)
bois de rose oil and oil of lemon grass (2.5% w/w each).

Candles were lit and placed in respective booths for 5 hours. The candles were then smothered and the dust was left exposed in the booths for 16 hours. The dust was then collected as for the controls and weighed.

During the 5 hour burn period approximately 27 g of each candle tested was burnt. This equated to a rate of 270 μl of deactivant dispersed per hour.

The collected samples were assayed by Der p1 ELISA to determine the allergen content. This was then related to the weight of dust that had been present in each sample. All the samples were multiplied up to compare the amount of allergen expected to be present in a 0.1 g sample of dust. The percentage differences between the control samples and the exposed samples were then obtained.

The Der p1 allergen reductions were as follows:
orange oil—67.8%
spearmint oil—37.7%
bois de rose oil—59.2%
frankincense—39.0%
oil of bergamot—44.7%
oil of lemon grass—48.0%
bois de rose oil and oil of bergamot (2.5% w/w each)—79.1%
bois de rose oil and oil of lemon grass (2.5% w/w each)—61.5%.

### EXAMPLE 5—HARD SURFACE/CANDLE

House dust was passed through a number of sieves and the fraction smaller than 53 μm was collected. 0.025 g of dust was placed in a small sieve to distribute it evenly over the test surface. The test surface was a PTFE coated metal tray of size 30 cm by 30 cm. The dust was applied to the tray by moving the sieve continuously over the surface while tapping the sieve. One half of the dust was then removed by suction onto an in-line filter and the weight recorded, this was the pretreatment control. The tray was then placed in a booth. Candles were prepared, of approximately 60 g before testing, with two wicks, and having a sterin:wax ratio of 2:10 (w/w) and 5% w/w of deactivant incorporated into the candle body by a process of melting, mixing and setting.

The deactivants were:
oil of bergamot and orange oil (2.5% w/w each)
oil of jasmine and orange oil (2.5% w/w each)
oil of jasmine (1% w/w) and orange oil (4% w/w)
oil of jasmine (4% w/w) and orange oil (1% w/w).

Candles were lit and placed in the booth for 5 hours. The candles were then smothered and the dust was left exposed in the booth for 16 hours. The dust was then collected as for the controls and weighed.

During the 5 hour burn period approximately 27 g of each candle tested was burnt. This equated to a rate of 270 μl of deactivant dispersed per hour.

The collected samples were assayed by Der p1 ELiSA to determine the allergen content. This was then related to the weight of dust that had been present in each sample. All the samples were multiplied up to compare the amount of allergen expected to be present in a 0.1 g sample of dust. The percentage differences between the control samples and the exposed samples were then obtained.

The Der p1 allergen reductions were as follows:
oil of bergamot and orange oil (2.5% w/w each)—47.9%
oil of jasmine and orange oil (2.5% w/w each)—75.1%
oil of jasmine (1% w/w) and orange oil (4% w/w)—82.0%
oil of jasmine (4% w/w) and orange oil (1% w/w)—82.0%.

1. A method of deactivating an allergen, the method comprising dispersing into an airspace containing or able to support said allergen an allergen-deactivating amount of an allergen-deactivating compound (hereinafter the “deactivant”) comprising one or more of the following compounds:
a citrus oil;
a mint oil;
bois de rose oil;
oil of jasmine;
frankincense;
oil of bergamot; and
oil of lemon grass.

2. A method as claimed in claim 1, wherein the deactivant is dispersed into the airspace as a vapour over an extended period, by the use of heat.

3. A method as claimed in claim 1 or 2, wherein the deactivant is dispersed via a wick dipped into a reservoir of the deactivant.

4. A method as claimed in any preceding claim, wherein the deactivant is provided as a water-in-oil emulsion containing up to 2% by weight of the deactivant.

5. A method as claimed in claim 1 or 2, wherein the deactivant is incorporated into a candle which is burnt in the space to be treated.

6. A method as claimed in claim 5, wherein the candle comprises at least 2% by weight of the deactivant (wt of deactivant/wt of combustible body of the candle).

7. A method as claimed in any preceding claim, wherein the deactivant is dispersed into the airspace on two or more occasions, separated by period(s) of no deactivant dispersal.

8. A method as claimed in any preceding claim, wherein the deactivant is a said oil in its as-extracted form.

9. A method as claimed in any preceding claim, and which employs as deactivant two or more of said compounds, wherein said compounds are co-dispersed into the airspace.

10. A method as claimed in claim 9, and employing one of the following deactivant combinations:
oil of bergamot and bois de rose oil;
oil of lemon grass and bois de rose oil;
a citrus oil and oil of bergamot;
a citrus oil and oil of jasmine.

11. Use in deactivating an allergen at a locus of one or more of the following materials:
a citrus oil;
a mint oil;
bois de rose oil;
oil of jasmine;
frankincense;
oil of bergamot;
oil of lemon grass.

12. Use of one or more of the following materials:
a citrus oil;
a mint oil;
bois de rose oil;
oil of jasmine;
frankincense;
oil of bergamot;
oil of lemon grass;
dispersed into an airspace in order to deactivate an allergen on a surface which is within that airspace.

13. An allergen deactivant, being one of the following:
a mixture of oil of bergamot and bois de rose oil;
a mixture of oil of lemon grass and bois de rose oil;
a mixture of a citrus oil and oil of bergamot;
a mixture of a citrus oil and oil of jasmine.
